(19) United States
(12) Patent Application Publication
(10) Pub. No.: US 2020/0094051 A1
(43) Pub. Date: Mar. 26, 2020

(54) CANCER TREATING DEVICE

(71) Applicants: ELECTRONICS AND TELECOMMUNICATIONS RESEARCH INSTITUTE, Daejeon (KR); KOREA INSTITUTE OF RADIOLOGICAL & MEDICAL SCIENCES, Seoul (KR)

(72) Inventors: Hyung Ju PARK, Suwon (KR); Dae-Sik LEE, Daejeon (KR); Jae-Sung KIM, Namyangju (KR); Yi Na YOON, Seoul (KR)

(21) Appl. No.: 16/576,472

(22) Filed: Sep. 19, 2019

(30) Foreign Application Priority Data
Sep. 20, 2018 (KR) 10-2018-0113213
Aug. 27, 2019 (KR) 10-2019-0105063

(51) Int. Cl. A61N 1/36; A61N 1/378; A61N 1/08

(52) U.S. Cl. CPC A61N 1/36002; B82Y 30/00; A61N 1/08; A61N 1/378

(57) ABSTRACT

Provided is a cancer treating device including a signal generator, a temperature sensor electrically connected to the signal generator, and a pair of electrodes which receive an AC voltage from the signal generator, wherein the signal generator is configured to generate an electric field between the electrodes so as to change orientations of ferroelectric particles inside a cancer cell, the temperature sensor measures a temperature around the cancer cell, and the signal generator may be configured to change the intensity of the electric field on the basis of the measured temperature.

### CROSS-REFERENCE TO RELATED APPLICATIONS

This U.S. non-provisional patent application claims priority under 35 U.S.C. § 119 of Korean Patent Application Nos. 10-2018-0113213, filed on Sep. 20, 2018, and 10-2019-0105063, filed on Aug. 27, 2019, the entire contents of which are hereby incorporated by reference.

### BACKGROUND

The present disclosure herein relates to a cancer treating device, and more particularly, to a cancer treating device using an electric field.

Cell division occurs when microtubules having dipole moments are coupled to each other by electric force to form spindles, and the generated spindles pull chromosomes arranged at the center of a cell toward both sides of the cell.

If a cancer cell is interfered with such cell division, treatment of cancers becomes possible through the suppression of cell division of the cancer cell.

### SUMMARY

The present disclosure provides a structure of a cancer treating device for effectively suppressing cell division of a cancer cell.

The object of the present disclosure is not limited to the aforesaid, but other objects not described herein will be clearly understood by those skilled in the art from descriptions below.

An embodiment of the inventive concept provides a cancer treating device including: a signal generator; a temperature sensor electrically connected to the signal generator; and a pair of electrodes which receive an AC voltage from the signal generator, wherein the signal generator is configured to generate an electric field between the pair of electrodes so as to change orientations of ferroelectric particles inside a cancer cell, the temperature sensor measures a temperature around the cancer cell, and the signal generator is configured to change an intensity of the electric field on the basis of the measured temperature.

In an embodiment, the electric field may have a frequency of about 10 KHz to about 50 KHz.

In an embodiment, the ferroelectric particles may have diameters of greater than about 0 nm and equal to or smaller than about 50 nm, and the ferroelectric particles may have any one of BaTiO₃ or SrTiO₃.

In an embodiment, the first electrode and the second electrode may include ferroelectrics.

In an embodiment, the ferroelectric particles are positioned inside cytoplasm of the cancer cell in an intermediate stage during a pseudo-division process of the cancer cell, and at least a portion of the chromosomes may not be coupled to centrosomes due to an electric field around the ferroelectrics.

In an embodiment, the ferroelectrics may be disposed between mutually facing cleavage furrows of the cancer cell in a final stage during the pseudo-division process of the cancer cell.

In an embodiment of the inventive concept, a cancer treating device includes: a first electrode and a temperature sensor on one surface of a first patch; a second electrode and a temperature sensor on one surface of a second patch; and a signal generator electrically connected to the first electrode and the second electrode, wherein the signal generator is configured to generate an electric field between the first electrode and the second electrode so as to change orientations of nanoparticle probes in the cancer cell, each of the first and second temperature sensors measures a temperature around the cancer cell, the signal generator is configured to change the intensity of the electric field on the basis of the measured temperature, and division of the cancer cell is suppressed according to the changed orientations of the nanoparticles.

In an embodiment, the nanoparticle probes may each include a ferroelectric particle, a plurality of biomarkers attached to the ferroelectric particle, wherein the biomarkers may target the cancer cell and a passivation film coated on the ferroelectric particle.

In an embodiment, the nanoparticle probes may move inside the cancer cell by the electric field.

In an embodiment of the inventive concept, the cancer treating device includes: a signal generator; a first electrode and a second electrode which face each other; a third electrode and a fourth electrode which face each other; and a temperature sensor electrically connected to the signal generator, wherein the first electrode and the second electrode receive a first AC voltage from the signal generator, the third electrode and the fourth electrode receive a second AC voltage from the signal generator, the signal generator generates a first electric field between the first electrode and the second electrode so as to change the orientation of the ferroelectric particles inside the cancer cell, the signal generator generates a second electric field between the third electrode and the fourth electrode so as to change an orientation of polar molecules inside the cancer cell, and the first electric field and the second electric field have mutually different frequencies.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a conceptual view of a cancer treating device according to an embodiment of the inventive concept;

FIG. 2A is an enlarged view of portion “aa” in FIG. 1;

FIG. 2B is an enlarged view of portion “bb” in FIG. 1;

FIG. 3A is a conceptual view illustrating an intermediate stage of a cell division process of a cancer cell;

FIG. 3B is a conceptual view illustrating a final stage of a cell division process of a cancer cell;

FIGS. 4A and 4B are conceptual views illustrating application examples of the inventive concept;

FIG. 5 is a conceptual view illustrating a cancer treating device according to an embodiment of the inventive concept;

FIG. 6A is a graph illustrating a relative number of cells remaining after applying an electric field;

FIG. 6B is a view illustrating cells remaining after applying an electric field;

FIG. 7A is a graph illustrating a relative number of colonies remaining after applying an electric field; and

FIG. 7B is a view illustrating colonies remaining after applying an electric field.

### DETAILED DESCRIPTION

Exemplary embodiments of the inventive concept will be described with reference to the accompanying drawings so as to sufficiently understand configurations and effects of the inventive concept. The inventive concept may, however, be embodied in different forms and should not be construed as limited to the embodiments set forth herein. Rather, these embodiments are provided so that this disclosure will be thorough and complete, and will fully convey the scope of the present invention to those skilled in the art. In the accompanying drawings, the dimensions of components are exaggerated for convenience in description, and the ratios of the components may be exaggerated or reduced.

Unless terms used in embodiments of the present invention are differently defined, the terms may be construed as meanings that are commonly known to a person skilled in the art. Hereinafter, the present invention will be described in detail by explaining preferred embodiments of the invention with reference to the attached drawings.

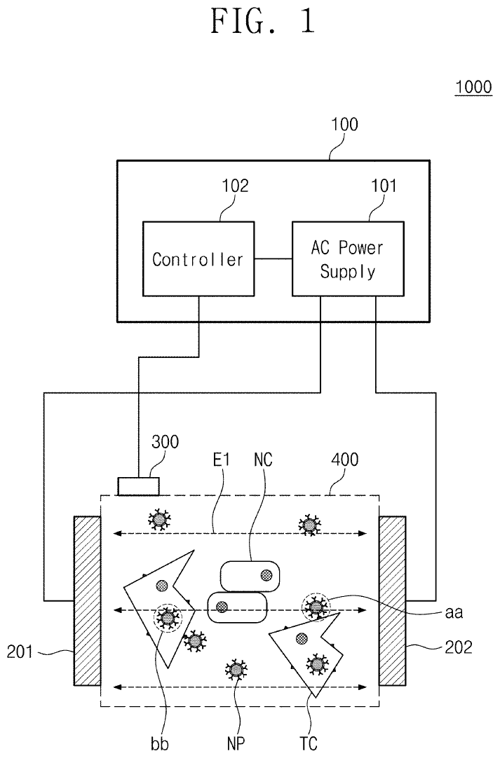

FIG. 1 is a conceptual view of a cancer treating device according to an embodiment of the inventive concept.

Referring to FIG. 1, a cancer treating device 1000 according to an embodiment of the inventive concept may include a signal generator 100, a first electrode 201, a second electrode 202, and a temperature sensor 300.

The signal generator 100 may include a controller 102 and an AC power supply 101. The controller 102 may determine the amplitude and frequency of an electric field E1 to be described later. The controller 102 may apply an AC voltage to the first electrode 201 and the second electrode 202 through the AC power supply 101.

The first electrode 201 and the second electrode 202 may include a ferroelectric material. The first electrode 201 and the second electrode 202 may include, for example, at least any one among PMN-PT, PMN-PZT or BaTiO₃.

The first electrode 201 and the second electrode 202 may be disposed so as to be spaced apart from each other with an affected area 400 therebetween. At least a portion of the first electrode 201 and at least a portion of the second electrode 202 may face each other.

An electric field E1 may be formed by an AC voltage between the first electrode 201 and the second electrode 202. The direction of the electric field E1 may continuously vary according to the amplitude and frequency of the AC voltage. The electric field E1 may have a frequency of about 10 KHz to about 500 KHz.

The affected area 400 may be a region in which cancer cells TC are present. Normal cells NC may also be present in the affected area 400. The affected area 400 may correspond to, for example, any one region among the brain, the breast, the lung, or the like where the cancer cells TC are present.

A plurality of nanoparticle probes NP may be injected around/into the cancer cells TC. The nanoparticle probes NP may enter the inside of a body through an eating action and move to the affected area 400.

The temperature sensor 300 may measure the temperature of the skin on the affected area 400 and provide temperature information to the controller 102. The temperature sensor 300 may adjust the intensity or the like of the electric field E1 through the controller 102 and the AC power supply 101 so as to maintain the temperature of the affected area 400 or prevent the temperature of the affected area 400 from rising to at least a certain temperature in order to prevent burn.

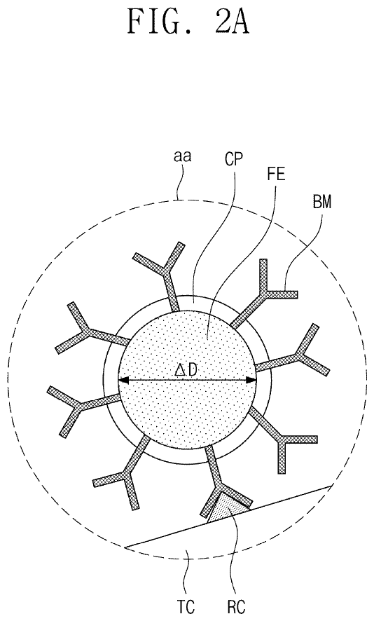

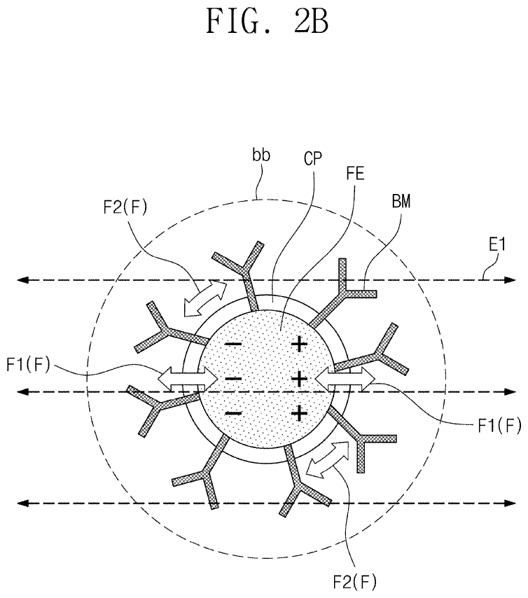

FIG. 2A is an enlarged view of portion “aa” in FIG. 1.

Referring to FIGS. 1 and 2A, each of the nanoparticle probes NP may include a ferroelectric particle FE and a plurality of biomarkers BM. If necessary, each of the nanoparticle probes NP may further include a passivation film CP.

The ferroelectric particle FE may have a spherical shape with a diameter ΔD of greater than about 0 nm and equal to or smaller than about 200 nm. As long as the ferroelectric characteristics are maintained, the smaller the diameter ΔD, the more effective, the ferroelectric particle FE may be. The ferroelectric particle FE may include at least one of BaTiO₃ or SrTiO₃.

The biomarkers BM may be attached to the ferroelectric particle FE. The nanoparticle probes NP may each be coupled, through the biomarkers BM, to a receptor RC on the surface of the cancer cell TC. The nanoparticle probe NP coupled to a receptor RC of the cancer cell TC may enter the inside of the cancer cell TC by phagocytosis of the cancer cell TC.

The passivation film CP may cover the surface of the ferroelectric particle FE. The passivation film CP may prevent a non-specific coupling reaction of the nanoparticle probe NP. The passivation film CP may include, for example, fetal bovine serum (FBS).

FIG. 2B is an enlarged view of portion “bb” in FIG. 1.

Referring to FIGS. 1 and 2B, an electric field E1 may be applied to the nanoparticle probe NP. When the electric field E1 is applied to the nanoparticle probe NP, a polarization phenomenon may occur inside the ferroelectric particle FE of nanoparticle probe NP. That is, according to the direction of the electric field E1, one portion of the ferroelectric particle FE may be charged with positive (+) charges and the other portion may be charged with negative (−) charges.

The direction of the electric field E1 may continuously vary due to an AC voltage. Once being polarized, the ferroelectric particle FE may maintain the polarized state due to the material characteristics thereof even when an electric field is not applied. When an electric field having another direction is applied in the polarized state, re-polarization may occur in the direction of the electric field.

When the direction of the electric field E1 varies, the orientation of the nanoparticle probe NP may vary according to the varied direction of the electric field E1. During the varying process of the electric field E1, the ferroelectric particle FE may receive an electrical force. The orientation variation F in this specification may include all of the frequency F1, the rotation F2, and the like of the nanoparticle probe NP.

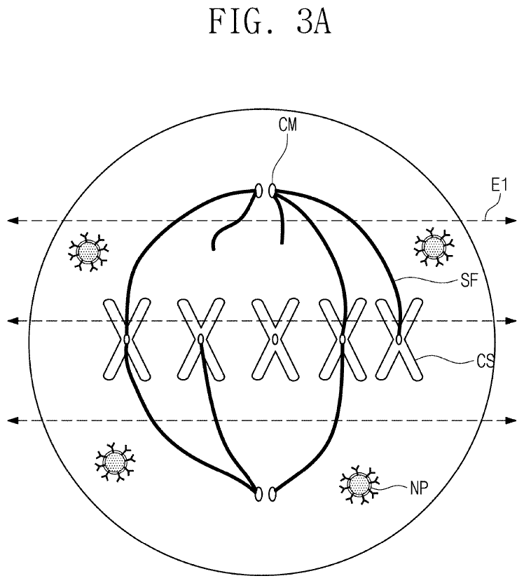

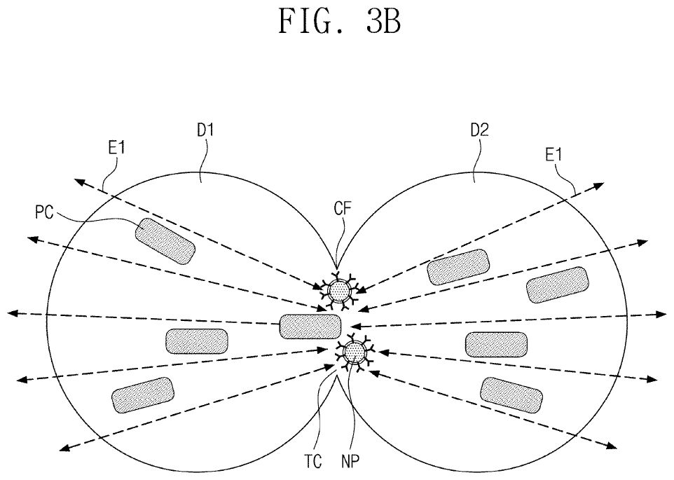

FIG. 3A is a conceptual view illustrating an intermediate stage of a cell division process of a cancer cell.

Referring to FIG. 3A, a polarized nanoparticle NP may be moved by an electrical attraction force so as to be adjacent to microtubules inside a spindle SF having a dipole moment. The nanoparticle probe NP may be located inside cytoplasm of a cancer cell TC. The nanoparticle probes NP may affect the cancer cell TC by electrical characteristics expressed by an electric field E1. For example, at least a portion of chromosomes may not be coupled to centrosomes by an electric field around the nanoparticle probes NP.

Spindles SF extending from the centrosomes CM may not be connected to the chromosomes CS due to steric hindrance of the nanoparticle probes NP. In addition, the spindles SF may not be connected to the chromosomes CS due to vibration or rotation of the nanoparticle probes NP.

In another embodiment, the orientations of the microtubules inside the spindles SF are varied by the electric field E1 and at least a portion of the spindles SF may be destroyed. The spindles SF may not be connected to the chromosomes CS by the destruction of the spindles SF.

FIG. 3B is a conceptual view illustrating a final stage of a cell division process of a cancer cell.

Referring to FIG. 3B, a cleavage furrows CF may be formed in the final stage of the division process of a cancer cell. Polar cell materials PC may be present in a first preliminary daughter cell D1 and a second preliminary daughter cell D2 with two mutually-facing cleavage furrows SF therebetween.

Comparing with FIG. 3A, the electric field E1 may be applied so as to have a non-uniform line of electric force. The nanoparticles probes NP may be moved by the electric field E1, and at least a portion of the nanoparticles probes NP may be moved between the two cleavage furrows CF. Contraction of the cancer call TC between the cleavage furrows CF may be prevented, and the cancer cell may be prevented from being divided into two daughter cells.

In another embodiment, at least a portion of the polar cell materials PC may be moved by the electric field E1 between the cleavage furrows CF. In this case, contraction of the cancer call TC between the cleavage furrows CF may be prevented, and the cancer cell may be prevented from being divided into two daughter cells.

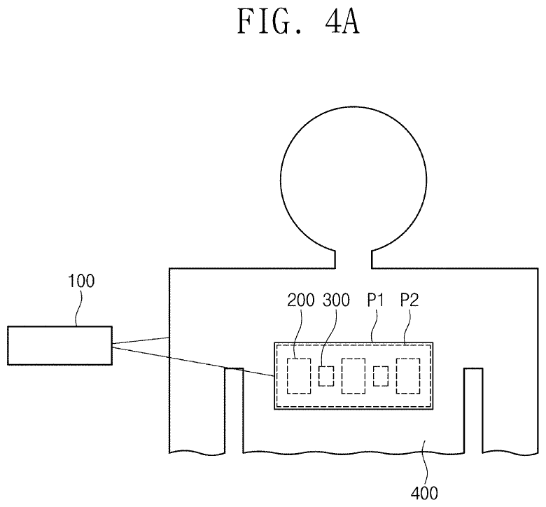

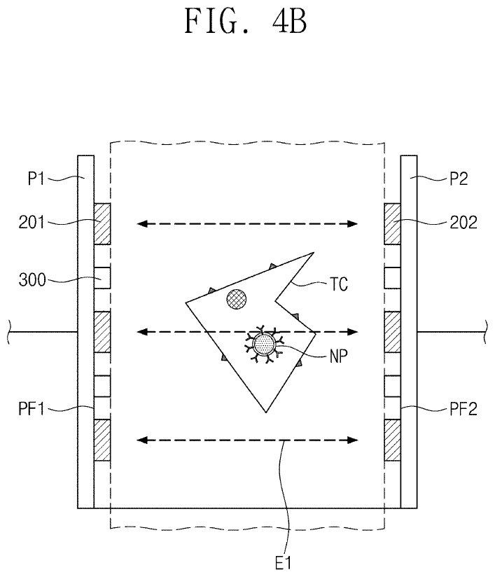

FIGS. 4A and 4B are conceptual views illustrating an application example of the inventive concept.

Referring to FIGS. 4A and 4B, a first patch P1 and a second patch P2 may be disposed with an affected area 400 therebetween. The first patch P1 may be adhered to the skin on the affected area 400 through an adhesive layer (not shown) provided on one surface PF1 of the first patch P1. The second patch P2 may be adhered to the skin (not shown) on the affected area 400 through an adhesive layer (not shown) provided on one surface PF2 of the second patch P2.

A plurality of first electrodes 201 and a plurality of temperature sensors 300 may be provided on the one surface PF1 of the first patch P1. A plurality of second electrodes 202 and a plurality of temperature sensors 300 may be provided on the one surface PF2 of the second patch P2. The first electrodes 201, the second electrodes 202, and the temperature sensors 300 may be electrically connected to a signal generator 100.

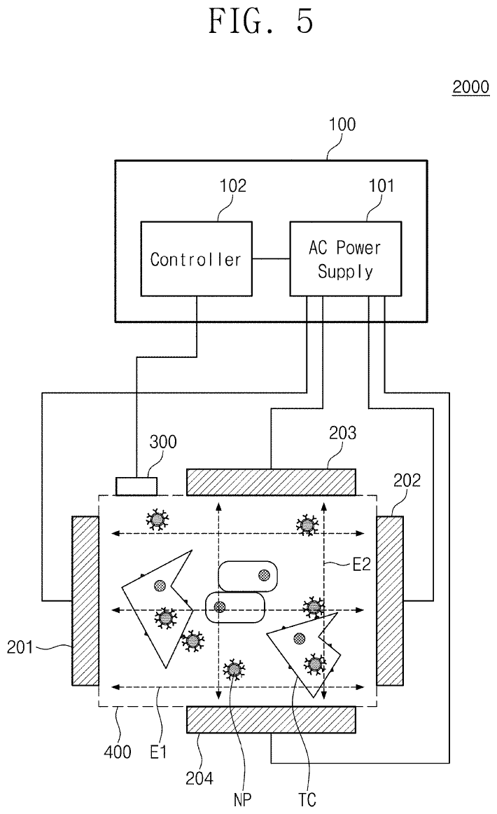

FIG. 5 is a conceptual view illustrating a cancer treating device according to an embodiment of the inventive concept. Since having been described in detail through FIG. 1, description on the cancer treating device will be omitted except for the matters described below.

Referring to FIG. 5, a cancer treating device 2000 according to an embodiment of the inventive concept may include a signal generator 100, a first electrode 201, a second electrode 202, a third electrode 203, a fourth electrode 204, and a temperature sensor 300.

The first electrode 201 and the second electrode 202 may be disposed so as to face each other with an affected area 400 therebetween. The third electrode 203 and the fourth electrode 204 may be disposed so as to face each other with the affected area 400 therebetween. According to another embodiment, any one of the third electrode 203 and the fourth electrode 204 may be omitted.

A first electric field E1 may be generated between the first electrode 201 and the second electrode 202. A second electric field E2 may be generated between the third electrode 203 and the fourth electrode 204. The first electric field E1 and the second electric field E2 may have amplitudes and/or frequencies different from each other.

The first electric field E1 may have a specific frequency that causes the orientation of the nanoparticles probes NP to vary. The frequency of the first electric field E1 may be smaller than the frequency of the second electric field E2. The frequency of the first electric field E1 may include the range from about 10 KHz to about 100 KHz.

As described above, the nanoparticle probes NP may suppress cell division of a cancer cell TC by the first electric field E1 due to prevention of the connection of the centrosomes and chromosomes through spindles in the cell division process and prevention of generation of daughter cells.

The second electric field E2 may have a specific frequency that causes the orientation of polar materials of the cancer cell TC to vary. The frequency of the second electric field E2 may include the range from about 100 KHz to about 500 KHz.

The spindles may be destroyed due to orientation of microtubules in the intermediate stage process of the division of the cancer cell TC using the second electric field E2. The cancer cell may not be separated into two daughter cells due to the movement of the polar cell materials between the cleavage furrows, the movement being caused by the second electric field E2.

### Comparative Example 1

A BT-549 breast cancer cell line was purchased from American Type Culture Collection (ATCC; Manassas, Va.) and prepared. BT-549 cells were cultured in RPMI (corning). About 10% of FBS and about 1% of penicillin/streptomycin were supplemented to the BT-549 cells. The BT-549 cells were maintained at about 37° C. in about 5% humidified CO₂ incubator.

The BT-549 cells were maintained for about 24 hours in an about-22 mm plastic cover slip (Thermo Fisher Scientific, MA, USA). Then, the cover slip was moved to a ceramic in-vitro dish (NovoCure, Haifa, Israel) by using an autoclaved forceps.

### Experimental Example 1

A dish was prepared in which cancer cells were cultured under the same condition as that in the comparative example.

Barium titanate nanoparticles (BTNP) having diameters of about 100 nm were purchased from US Research Nanomaterials Inc. (TX, USA) and dispersed in ethanol without additional purification, and aggregates were dispersed by ultrasonic processing. By attaching biomarkers, nanoparticle probes having concentration of greater than about 0 second and equal to or smaller than about 20 μg/ml were formed. By adding about 5% of FBS was added before treating to the cancer cell, the BTNP were coated with protein corona. Nanoparticle probes were added to the dish in which the cancer cells are cultured.

### Experimental Example 2

Except for using BTNP having diameters of about 200 nm, an experiment was performed in the same manner as the experimental example 1. The BTNP having diameters of about 200 nm were purchased from US Research Nanomaterials Inc. (TX, USA).

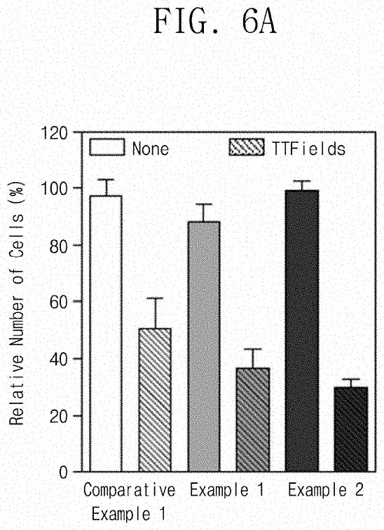

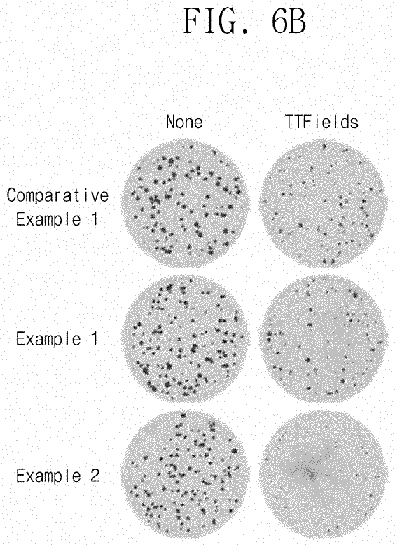

FIG. 6A is a graph illustrating a relative number of cells remaining after applying an electric field. FIG. 6B is a view illustrating cells remaining after applying an electric field. The views in FIG. 6B are views of measurements using a VersaMax Microplate Reader (Molecular device, CA, USA) of about 450 nm.

An electric field was applied for three days in a cancer cell culturing dish at a current of about 150 mA with about 150 KHz. The temperature was maintained at about 37° C. by using an inviotro system refrigeration incubator (ESCO Technologies, TX, USA).

In order to measure the number of live cells with the same volume, BD Accuri™ C6 (BD biosciences, CA, USA) was used to perform the experiment. The cells are dyed with about 500 μg/mL of propidium iodide (PI; Sigma-Aldrich), and the number of cells in a PI-negative group was calculated with respect to a volume of about 100 μl. Referring to FIGS. 6A and 6B, when an electric field is not applied (none), the number of cancer cells somewhat decreases in experimental example 1 compared to that in comparative example 1, but the number of cancer cells in experimental example 2 is almost the same as that in comparative example 1.

When an electric field is applied (TTFields), the number of cancer cells further decreases in all of experimental example 1, experimental example 2, and comparative example 1, compared to that in which an electric field is not applied (none).

In particular, when an electric field is applied, it may be confirmed that in experimental example 1 and experimental example 2, in which nanoparticle probes are added, a smaller number of cells remain than that in comparative example 1.

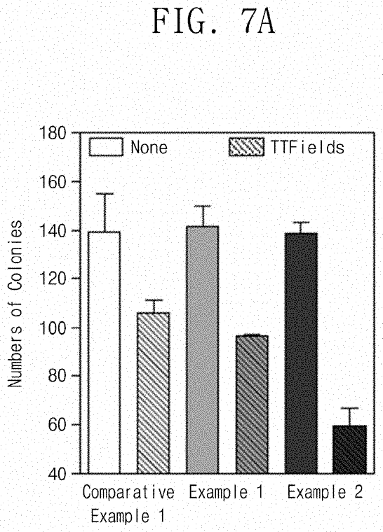

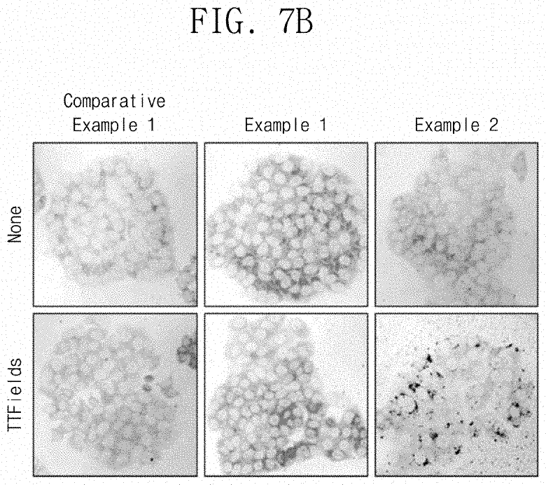

FIG. 7A is a graph illustrating a relative number of colonies remaining after applying an electric field. FIG. 7B is a view illustrating colonies remaining after applying an electric field.

An electric field was applied for about three days in a cancer cell culturing dish at a current of about 150 mA with about 150 KHz. A cover slip was moved to a 6-well plate after applying an electric field, and incubation was performed at about 37° C. Colonies were fixed after about seven days, dyed with crystal violet (Sigma-Aldrich) of about 1% and a methanol solution of about 40%, and then, the number of colonies was counted.

Referring to FIGS. 7A and 7B, when the electric field is not applied (none), the number of colonies somewhat increases in experimental example 1 compared to that in comparative example 1, and the number of colonies in experimental example 2 is almost the same as that in comparative example 1.

When an electric field is applied (TTFields), the number of colonies decreases in all of experimental example 1, experimental example 2, and comparative example 1, compared to that in which an electric field is not applied.

In particular, when an electric field is applied, it may be understood that in experimental example 1 and experimental example 2, in which nanoparticles probes were added, a smaller number of colonies remain than that in comparative example 1.

In a cancer treating device according to an embodiment of the inventive concept, nanoparticle probes including ferroelectric are injected to a cancer cell, and an electric field having a specific frequency is applied, and thus, cell division of the cancer cell may effectively be suppressed.

As described above, cell division of a cancer cell may effectively be suppressed through a cancer treating device according to the inventive concept.

So far, embodiments of the inventive concept have been described.

However, those skilled in the art could understand that the inventive concept may be implemented in other specific forms without changing the technical spirit and indispensible characteristics. Thus, the above-disclosed embodiments are to be understood illustrative and not restrictive.

What is claimed is:

1. A cancer treating device comprising:
a signal generator;
a temperature sensor electrically connected to the signal generator; and
a pair of electrodes which receive an AC voltage from the signal generator, wherein the signal generator is configured to generate an electric field between the pair of electrodes so as to change orientations of ferroelectric particles inside a cancer cell,
the temperature sensor measures a temperature around the cancer cell, and
the signal generator changes an intensity of the electric field on the basis of the measured temperature.

2. The cancer treating device of claim 1, wherein the electric field has a frequency of about 10 KHz to about 500 KHz.

3. The cancer treating device of claim 1, wherein
the ferroelectric particles have diameters of greater than about 0 nm and equal to or smaller than about 50 nm, and
the ferroelectric particles comprise at least any one of BaTiO₃ or SrTiO₃.

4. The cancer treating device of claim 1, wherein each of the electrodes comprises ferroelectrics.

5. The cancer treating device of claim 1, wherein
the ferroelectric particles are located inside cytoplasm of the cancer cell in an intermediate stage during a pseudo-division process of the cancer cell, and
at least a portion of the chromosomes of the cancer cells are not coupled to centrosomes of the cancer cells due to an electric field around the ferroelectrics.

6. The cancer treating device of claim 1, wherein the ferroelectrics are disposed between mutually facing cleavage furrows of the cancer cell in a final stage during the pseudo-division process of the cancer cell.

7. A cancer treating device comprising:
a first electrode and a temperature sensor on one surface of a first patch;
a second electrode and a temperature sensor on one surface of a second patch; and
a signal generator electrically connected to the first electrode and the second electrode, wherein
the signal generator is configured to generate an electric field between the first electrode and the second electrode so as to change orientations of nanoparticle probes in the cancer cell,
each of the first and second temperature sensors is configured to measure a temperature around the cancer cell,
the signal generator is configured to change an intensity of the electric field on the basis of the measured temperature, and
division of the cancer cell is suppressed according to the changed orientations of the nanoparticles.

8. The cancer treating device of claim 7, wherein the nano-particle probes each comprises:
a ferroelectric particle,
a plurality of biomarkers attached to the ferroelectric particle, wherein the biomarkers target the cancer cell, and
a passivation film coated on the ferroelectric particle.

9. The cancer treating device of claim 7, wherein the nanoparticle probes move inside the cancer cell by the electric field.

10. A cancer treating device may include:
a signal generator;
a first electrode and a second electrode which face each other;
a third electrode and a fourth electrode which face each other; and
a temperature sensor electrically connected to the signal generator, wherein
the first electrode and the second electrode receive a first AC voltage from the signal generator,
the third electrode and the fourth electrode receive a second AC voltage from the signal generator,
the signal generator generates a first electric field between the first electrode and the second electrode so as to change the orientations of the ferroelectric particles inside the cancer cell,
the signal generator generates a second electric field between the third electrode and the fourth electrode so as to change orientations of polar molecules inside the cancer cell, and
the first electric field and the second electric field have mutually different frequencies.
